(19) United States
(12) Patent Application Publication
(10) Pub. No.: US 2021/0204964 A1
(43) Pub. Date: Jul. 8, 2021

(54) SURGICAL HANDLE

(71) Applicant: FIELD ORTHOPAEDICS PTY LTD, Red Hill Brisbane, Queensland (AU)

(72) Inventors: Shanthan PATHER, Red Hill Brisbane, Queensland (AU); Christopher Arnold JEFFERY, Red Hill Brisbane, Queensland (AU)

(21) Appl. No.: 17/059,057

(22) PCT Filed: May 20, 2019
(86) PCT No.: PCT/AU2019/050485

§ 371 Date: Nov. 25, 2020

(30) Foreign Application Priority Data
May 25, 2018 (AU) 2018901837

(51) Int. Cl. A61B 17/16; A61B 17/88

(52) U.S. Cl. CPC A61B 17/162; A61B 2017/00464; A61B 17/8875

(57) ABSTRACT

A surgical handle for interfacing with a replaceable tool for use during a surgical procedure includes a head that is held in the palm of the hand and a gripping member held by the fingers. Turning of the gripping member results in a shaft of the replaceable tool being driven. Between the head and the gripping member is a movable sleeve that is pulled upwards in order to uncouple the handle from the shaft and is released in order to couple the shaft with the handle. The coupling between the shaft and the handle includes a circumferentially positioned retaining member that is pushed by the sleeve into engagement with a detent on the shaft.

### TECHNICAL FIELD

The present disclosure relates to the field of surgical instrumentation, and more particularly relates to a surgical handle for use with surgical drivers such as surgical screws or surgical bits for use by surgeons during various medical procedures.

### BACKGROUND

The discussion of any prior art documents, techniques, methods or apparatus is not to be taken to constitute any admission or evidence that such prior art forms, or ever formed, part of the common general knowledge.

Various types of fasteners are used to engage implants and other devices to bone. During orthopaedic surgery, bone screws are commonly used to attach plates, rods and other types of implants and devices to one or more bones. In some instances, a relatively high degree of precision is required to engage the bone screws in the proper position and orientation. Various types of driving bits are also frequently used by orthopaedic surgeons.

Surgical drivers are an essential tool in orthopaedic surgery. Issues with surgical drivers occur from the frequent use and wear and tear. The driver bits can get worn out and can be more prone to stripping during use. To address issues associated with bit wear and the patient risk associated with drive interface stripping, quick connect interfaces (or simply “quick connects”) have become widely popular in surgical instruments for connecting a shaft of a replaceable tool such as a bit, to a drive mechanism. The inclusion of quick connect interface into driver handles that allow the driver bits to be exchanged whilst allowing the reuse of the majority of the body (handle) is beneficial for greater sustainability and providing better value for end users.

A typical AO quick connect interface is configured to receive an end of a shaft having a substantially D-shaped profile. A flat portion defined by the D-shaped profile of engages another flat portion (referred to herein as the “AO flat) formed in an inner cavity of the AO quick connect interface. This engagement prevents rotation of the shaft with respect to the interface.

The issue with the use of the prior art AO quick couple interface drivers that are used in hand surgery is that a different hand grip is used where a surgeon will adopt a “tripod” grip to hold the driver handle. This grip is used for the purpose of increasing precision. However, with at least some prior art drivers, the quick couple mechanism is simply added on to the end of the handle making the driver too long for safe and effective use. The issue with such prior art driver handles is that the gripping portion for the screw driver is situated away from the connecting interface. As a result, using a tripod grip for a higher degree of precision is difficult to achieve. The requirement for achieving precision becomes even greater when surgeons are working with small surgical elements (such as but not limited to bone screws that have diameters in the range of 1 to 3 mm). In view of the above, there is a need to provide an improved driver handle that addresses some of the shortcomings of the prior art.

### SUMMARY OF THE INVENTION

In one aspect, the disclosure provides a surgical handle for interfacing with a replaceable tool for use during a surgical procedure, the surgical handle comprising:

a body comprising a proximal end portion having a cavity for receiving and releasably engaging a shaft of the replaceable tool;

a retaining member configured for being positioned circumferentially relative to the shaft received in the cavity, the retaining member being arranged to engage a detent on the shaft and retain the shaft in the cavity in a coupled configuration;

a gripping member having an outer surface adapted for being gripped by a surgeon during the surgical procedure to drive the shaft, the gripping member comprising a bore extending therethrough between two ends of the gripping member for receiving and engaging the body such that turning the gripping member results in the shaft being driven in a clockwise or anticlockwise direction in the coupled configuration; and

a movable sleeve member adapted to be received in between the gripping member and the body, wherein a first movement of the sleeve member pushes the retaining member into engagement with the detent on the shaft and limits movement of the retaining member thereby coupling the shaft with the body in the coupled configuration; and wherein a second movement of the sleeve releases the retaining member from the detent in the shaft.

In another aspect, the disclosure provides a surgical handle for interfacing with a replaceable tool for use during a surgical procedure, the surgical handle comprising:

a body comprising:
a proximal end portion having a cavity for receiving and releasably engaging a shaft of the replaceable tool; and
a distal end portion located away from the proximal end portion;

a retaining member moveable relative to the body configured for being positioned circumferentially relative to the shaft received in the cavity, the retaining member being arranged to be movable between a coupled configuration and a released configuration wherein in the coupled configuration the retaining member engages a detent on the shaft and retains the shaft in the cavity;

a gripping member having an outer surface adapted for being gripped by a surgeon during the surgical procedure to drive the shaft, the gripping member comprising a bore extending therethrough between two ends of the gripping member for receiving and engaging the body such that turning the gripping member results in the shaft being driven in a clockwise or anticlockwise direction in the coupled configuration; and

a movable sleeve member adapted to be received on the body such that the sleeve member is movable relative to a distal end of the body wherein a first movement of the sleeve member relative to the distal end of the body pushes the retaining member into engagement with the detent on the shaft and limits movement of the retaining member thereby coupling the shaft with the body in the coupled configuration; and wherein a second movement of the sleeve relative to the distal end of the body releases the retaining member from the detent in the shaft.

In an embodiment, a first end of the gripping member receives and engages the proximal end portion of the body

In an embodiment, the sleeve member is adapted to be telescopically received in between the gripping member and the body.

In an embodiment, the sleeve member is axially movable (relative to the body) such that the first movement comprises an inward axial movement of the sleeve member in a direction towards the proximal end portion of the retaining member effecting the retaining member to be pushed into engagement with the detent in the coupled configuration and wherein the second movement comprises an outward axial movement of the sleeve in a direction away from the proximal end portion of the retaining member effecting the release of the retaining member from the detent in the shaft.

In an embodiment, the surgical handle further comprises a biasing arrangement for biasing the movable sleeve member to apply a biasing force on the sleeve in the inward axial direction.

In an embodiment, the gripping member is adapted to be fastened to the proximal end portion of the body for preventing relative movement between the proximal portion of the body and the gripping member during use.

In an embodiment, upon the first movement of the sleeve member, an end portion of the sleeve member pushes the retaining member into the detent.

In a further embodiment, the end portion of the sleeve member comprises a cam portion such that upon the first movement the cam portion pushes the retaining member into the detent of the shaft of the replaceable tool.

Preferably, the end portion of the sleeve member comprises an angled surface forming the cam for engaging and pushing the retaining member into the detent during the first movement of the sleeve.

More preferably, upon the inward axial movement of the sleeve member, the end portion of the sleeve member pushes the retaining member into the detent of the shaft.

In an embodiment, the angled surface of the sleeve member is adapted for engaging and pushing the retaining member into the detent during the inward axial movement of the sleeve.

In an embodiment, the retaining member comprises a spherical member positioned in the body.

In an embodiment, the retaining member is biased by a biasing member to bias the retaining member in an inwardly direction towards the detent.

In an embodiment, an inner surface of the gripping member defines a recessed portion for receiving and engaging the sleeve member.

In an embodiment, the surgical handle further comprises a stop for stopping and thereby limiting the first movement of the sleeve member.

Preferably, the stop limits the inward axial movement of the sleeve member.

In an embodiment, the stopping member is positioned adjacent the recessed portion to stop and thereby limit inward axial movement of the sleeve member along the recessed portion.

In an embodiment, a distal portion of the body extends outwardly from an upper portion of the sleeve member.

In a further embodiment, the surgical handle further comprises a head mounted onto the distal portion of the body. Preferably, the head further comprises a cap is arranged to be movable relative to the distal portion of the body and/or the head. More preferably, the cap is arranged to rotate relative to the distal portion of the body.

In an embodiment, an in-use upper portion of the sleeve comprises an outwardly divergent section with a greater diameter relative to the diameter of an in-use lower portion of the sleeve that is received in between the body and the gripping member.

In an embodiment, the gripping member is elongate and comprises a middle portion extending between two end portions wherein cross-sectional diameter of the middle portion is greater than the cross sectional diameter of any one of the end portions.

In an embodiment, the inner cavity of the body is adapted to receive an end of the shaft having a D-shaped profile such that a flat portion of the shaft engages a corresponding flat portion along the inner surface defining the cavity of the body.

### BRIEF DESCRIPTION OF THE DRAWINGS

The Detailed Description is not to be regarded as limiting the scope of the preceding Summary of the Disclosure in any way. The Detailed Description will make reference to a number of drawings as follows:

FIG. 1 illustrates a first sectional view of a surgical handle 100 in accordance with an embodiment of the disclosure;

FIG. 2 illustrates a second sectional view of a surgical handle 100 in accordance with an embodiment of the disclosure;

FIG. 2A illustrates an enlarged view of the inset from FIG. 2 showing the surgical handle 100 in an uncoupled configuration whereby the shaft is not locked into position by the retaining member 120;

FIG. 2B illustrates another enlarged view showing the surgical handle 100 in a coupled configuration whereby the shaft is locked into position by the retaining member 120; and

FIG. 3 illustrates a side perspective view of the surgical handle 100.

### DETAILED DESCRIPTION OF THE DRAWINGS

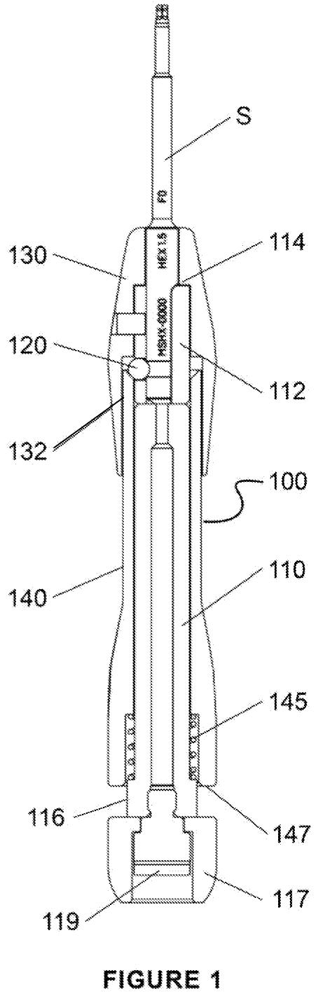

Referring to FIGS. 1 to 3, a surgical handle 100 for driving threaded members such as surgical screws (not shown) during surgical procedures is illustrated. The surgical handle 100 comprises an elongate body 110 having a proximal end portion 112 that includes a cavity 114 for receiving and releasably engaging a shaft S of a replaceable tool (such as drill bit). The preferred embodiment illustrates a quick connect interface that has been provided in the cavity 114 for receiving an end of the shaft S having a substantially D-shaped profile. A flat portion defined by the D-shaped profile of the shaft S typically engages another flat portion (referred to herein as the “AO flat”) formed along an inner wall of cavity 114 to prevent rotation of the shaft with respect to the body 110. It is important to note that the disclosure is in no way limited to use with shafts having a D-shaped profile. Accordingly, shafts with other profiles and shapes may also be used with alternative embodiments of the present disclosure.

Throughout the specification, the term “proximal” refers to a portion of the surgical handle 100 (a coupling end of the surgical handle 100) that is located in relatively close proximity to the shaft S of the replaceable tool and term “distal” refers to a portion of the surgical handle 100 that is located away from the shaft S.

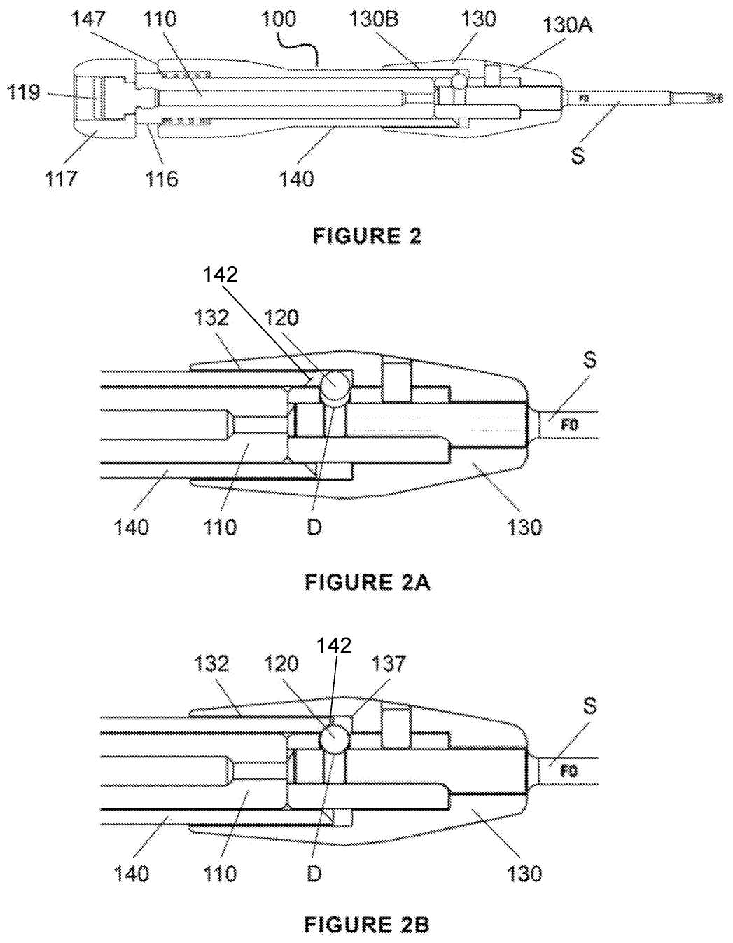

A retaining member in the form of a spring loaded ball 120 is positioned circumferentially relative to the shaft S received in the cavity 114. The spring loaded ball 120 may be biased by a biasing member such as a spring to engage and retain the shaft S in the cavity. The biasing force acting on the retaining member 120, in a neutral position, lodges the biasing member in a detent D (see FIGS. 2A and 2B) provided at or adjacent to an end portion of the shaft S. The bias on the retaining member 120 pushes a portion of the retaining member 120 into the detent D and assists in retaining the shaft S in the cavity 114 in a coupled configuration.

A gripping member 130 having an outer surface adapted for being gripped by a surgeon during the surgical procedure is used to drive bone screws by driving with the shaft S. The gripping member 130 comprises an elongate configuration with a bore extending there-through for housing and engaging the proximal portion 112 of the body 110 that receives and engages the shaft S. The gripping member 130 comprises a bulging middle section with a greater diameter when compared with end portions 130A and 130B with a relative smaller diameter. The inner surface of the gripping member 130 engages and mates with the proximal portion 112 of the body 110 such that any turning of the gripping member 130 by the surgeon in a clockwise or anti-clockwise direction (whilst the shaft S is engaged and locked into position) results in the rotational motion of proximal body portion 112 and results in the coupled shaft S (engaged with the body 110) to be driven in the clockwise or anti-clockwise direction. The location of the gripping member 130 at a proximal end of the surgical handle 100 is a very important function because such a configuration allows the surgeon to adopt a tripod grip (as explained in earlier sections) and achieve a high level of precision during surgeries.

The provision of a movable sleeve member 140 allows the surgeon to easily release or unlock the shaft S from the body 110 by a single handed operation. In the preferred embodiment, the sleeve member 140 is adapted to be telescopically received in between the gripping member 130 and the body 110. The inner surface of the gripping member 130 defines a recess 132 extending from a distal end of the gripping member 130 to a substantially central inner location of the gripping member 130 that accommodates the sleeve member 140 in between the gripping member 130 and the body 110 (as shown most clearly in FIGS. 2A and 2B). The movable sleeve member 140 can be moved such that a first movement of the sleeve member 140 results in coupling of the shaft S with the body 110 of the handle 100 and a second movement of the sleeve member 140 results in the shaft S being released from the cavity 114 of the body 110. As will be explained in the foregoing sections, the preferred embodiment of the surgical handle 100 provides a sleeve member 140 that is axially movable such that the first movement comprises an inward axial movement of the sleeve member effecting a biased retaining member 120 to be pushed into engagement with the detent D in the coupled configuration and wherein the second movement comprises outward axial movement of the sleeve 140 effecting the release of the biased retaining member 120 from the detent D in the shaft S. However, in other embodiments, the sleeve member 140 may undergo additional or alternative types of movements (such as but not limited to rotational or twisting movements) for allowing coupling and uncoupling of the releasable tool shaft S from the handle 100.

It must be understood that, as previously discussed, the sleeve member 140 may also be provided (in alternative embodiments) in the form of an incomplete or discontinuous sleeve 140′ (not shown) with a receiving portion or gate that can accommodate the retaining member 120 such that when a first twisting or torsional movement is imparted to the modified sleeve 140′, the retaining member 120 engages and retains the shaft S and in a second twisting or torsional movement, the retaining member 120 is released from the gate of the modified sleeve member 140′.

The sleeve member 140 is biased by a spring 145 positioned at an upper end of the surgical handle 100. In a neutral position (when no external force is applied by the surgeon), the sleeve 140 is pushed into the recess 132 until a lower end portion 142 (See FIGS. 2A and 2B) of the sleeve 140 contacts a stop 137. The lower end portion 142 includes a cam having an angled surface that is adapted to contact the spherical retaining member 120 such that upon pushing the sleeve 140 in an inwardly direction, the cam portion at the lower end of the sleeve 140 urges against the spherical retaining member 120 and the movement of the sleeve member 140 pushes the retaining member 120 into the detent D. The springs 145 apply a positive bias on the sleeve member 140 thereby maintaining the sleeve 140 in the inward position (as shown in FIG. 2B) and also keeping the shaft S locked within the cavity 114 of the body 110.

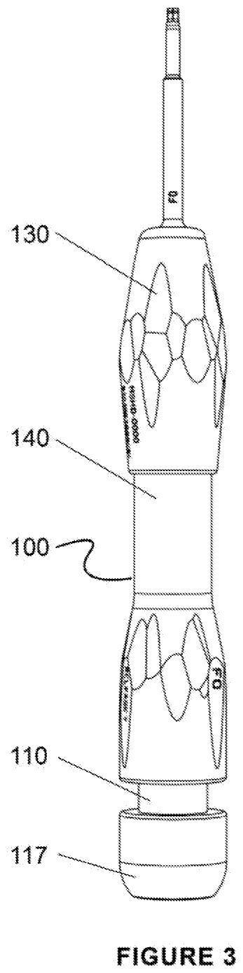

When the surgeon wishes to change or replace the tool, the shaft S may be released from the inner cavity 114 of the body 110 by pulling the sleeve 140 in an axially outward direction relative to the gripping member 130 as shown in FIG. 2A. The outward axial movement of the sleeve 140 results in the spring loaded spherical retaining member 120 to become dislodged out of the detent D thereby allowing the shaft S to be released from the inner cavity. It is also important to note that a distal head portion 117 of the body 110 extends outwardly from an upper portion of the sleeve 140. The aforementioned configuration of the movable sleeve member 140 in combination with the outwardly extending head portion 117 allows the surgeon to move the sleeve 140 in an upward direction by placing their thumb against the head portion 117 (See FIGS. 2 and 3). Advantageously, a distal portion 116 of the body 110 extends outwardly from an upper portion 147 of the sleeve member 140 and the head portion 119 is mounted onto the distal portion 116 of the body 110. In the preferred embodiment, the head portion 119 comprises a shank which is received into an opening in the distal portion 116 of the body 110. The head also includes an outer head cap 117.

The provision of the head 119 with a thumb receiving cap 117 allows the surgeon to place their thumb firmly against the cap 117 and apply a pushing force towards the head 119 (using the thumb) and the body 110 whilst applying a pulling force on the sleeve 140 (using the remaining fingers) to effect outward axial movement of the sleeve 140 towards the head 119. During typical use, the pulling force on the sleeve 140 needs to be sufficiently high to overcome the biasing force applied by the spring 145 positioned between the sleeve member 140 and the body 110 and effect an outward axial movement that results in the shaft S being uncoupled from the tool 100 (as previously discussed). Such a configuration allows the surgeon to uncouple the shaft S of the releasable tool from the surgical handle 100 by using a one handed operation or manipulation. Unlike some prior art surgical handles that may require bimanual operation, the surgical handle 100 can be operated with one hand and provides an improvement over at least some prior art surgical handles.

The head cap 117 is allowed to rotate freely relative to the head 119 and the distal portion 116 of the body 110. Such an arrangement allows the user to place the head cap 117 against their palm whilst gripping the surgical handle 100 at the gripping member 130. As a result, when a surgeon is turning the gripping member 130 in a clockwise or anticlockwise direction, the movement of the surgical handle's body 110 and the gripping member 130 does not effect any movement in the head cap 117 (which is held against the palm of the user). As a result, the freely rotating head cap 117 makes it easier for the surgeon to hold the head cap 117 against the palm firmly whilst also turning the surgical handle 100 during surgery.

In compliance with the statute, the disclosure has been described in language more or less specific to structural or methodical features. The term “comprises” and its variations, such as “comprising” and “comprised of” is used throughout in an inclusive sense and not to the exclusion of any additional features.

It is to be understood that the disclosure is not limited to specific features shown or described since the means herein described comprises preferred forms of putting the disclosure into effect.

The disclosure is, therefore, claimed in any of its forms or modifications within the proper scope of the appended claims appropriately interpreted by those skilled in the art.

1. A surgical handle for interfacing with a replaceable tool for use during a surgical procedure, the surgical handle comprising:
a body comprising:
a proximal end portion located at an in-use lower end of the surgical handle, the proximal end portion having a cavity for receiving and releasably engaging a shaft of the replaceable tool; and
a distal end portion located away from the proximal end portion at an in-use upper portion of the surgical handle;
a retaining member configured for being positioned circumferentially relative to the shaft received in the cavity, the retaining member being arranged to engage a detent on the shaft and to retain the shaft in the cavity in a coupled configuration;
a gripping member having an outer surface adapted for being gripped by a surgeon during the surgical procedure to drive the shaft, the gripping member comprising a bore extending therethrough between two ends of the gripping member for receiving and engaging the body such that turning the gripping member results in the shaft being driven in a clockwise or anticlockwise direction in the coupled configuration; and
a movable sleeve member adapted to be received in between the gripping member and the body wherein an in-use upper portion of the sleeve member extends beyond the gripping member towards the distal end of the body such that in at least one operable configuration, the distal portion of the body extends outwardly from the upper portion of the sleeve member, and
wherein a first movement of the sleeve member relative to the distal end of the body pushes the retaining member into engagement with a detent on the shaft and limits movement of the retaining member, thereby coupling the shaft with the body in the coupled configuration; and
wherein a second movement of the sleeve member relative to the distal end of the body releases the retaining member from the detent in the shaft.

2. A surgical handle for interfacing with a replaceable tool for use during a surgical procedure, the surgical handle comprising:
a body comprising:
a proximal end portion located at an in-use lower end of the surgical handle, the proximal end portion having a cavity for receiving and releasably engaging a shaft of the replaceable tool; and
a distal end portion located away from the proximal end portion at an in-use upper portion of the surgical handle;
a retaining member moveable relative to the body configured for being positioned circumferentially relative to the shaft received in the cavity, the retaining member being arranged to be movable between a coupled configuration and a released configuration wherein in the coupled configuration, the retaining member engages a detent on the shaft and retains the shaft in the cavity;
a gripping member having an outer surface adapted for being gripped by a surgeon during the surgical procedure to drive the shaft, the gripping member comprising a bore extending therethrough between two ends of the gripping member for receiving and engaging the body such that turning the gripping member results in the shaft being driven in a clockwise or an anticlockwise direction in the coupled configuration; and
a movable sleeve member adapted to be received on the body such that an in-use upper portion of the sleeve member extends beyond the gripping member towards the distal end of the body such that the upper portion of the sleeve member is movable relative to the distal end of the body,
wherein a first movement of the upper portion of the sleeve member relative to the distal end of the body pushes the retaining member into engagement with the detent on the shaft and limits movement of the retaining member thereby coupling the shaft with the body in the coupled configuration; and
wherein a second movement of the upper portion of the sleeve member relative to the distal end of the body releases the retaining member from the detent in the shaft.

3. The surgical handle in accordance with claim 1 wherein a first end of the gripping member receives and engages the proximal end portion of the body.

4. The surgical handle in accordance with claim 1 wherein the sleeve member is adapted to be telescopically received in between the gripping member and the body.

5. The surgical handle in accordance with claim 1 wherein the sleeve member is axially movable such that the first movement comprises an inward axial movement of the sleeve member, in a direction towards the proximal end portion of the retaining member, effecting the retaining member to be pushed into engagement with the detent in the coupled configuration and wherein the second movement comprises an outward axial movement of the sleeve, in a direction away from the proximal end portion of the retaining member, effecting the release of the retaining member from the detent in the shaft.

6. The surgical handle in accordance with claim 1 further comprising a biasing arrangement for biasing the movable sleeve member to apply a biasing force on the sleeve in an inward axial direction towards the gripping member.

7. The surgical handle in accordance with claim 1 wherein the gripping member is adapted to be fastened to the proximal portion of the body for preventing relative movement between the proximal portion of the body and the gripping member during use.

8. The surgical handle in accordance with claim 1 wherein upon the first movement of the sleeve member, an end portion of the sleeve member pushes the biasing member into the detent and wherein the end portion of the sleeve member comprises a cam portion such that upon the first movement the cam portion pushes the retaining member into the detent of the shaft of the replaceable tool.

9. (canceled)

10. The surgical handle in accordance with claim 8 wherein the end portion of the sleeve member comprises an angled surface for engaging and pushing the biasing member into the detent during the first movement of the sleeve.

11. The surgical handle in accordance with claim 7 wherein upon the first movement of the sleeve member, the end portion of the sleeve member pushes the retaining member into the detent of the shaft.

12. (canceled)

13. The surgical handle in accordance with claim 1 further comprising a biasing member for biasing the retaining member in an inwardly direction towards the detent.

14. The surgical handle in accordance with claim 1 wherein an inner surface of the gripping member defines a recessed portion for receiving and engaging an in-use lower portion of the sleeve member.

15. The surgical handle in accordance with claim 14 wherein the recessed portion is adapted to receive and engage the retaining member whereby the first movement of the sleeve member results in the retaining member being uncoupled from the recessed portion of the gripping member.

16. The surgical handle in accordance with claim 14 further comprising a stopping member configured for stopping and thereby limiting inward axial movement of the sleeve member, whereby the stopping member is positioned adjacent to the recessed portion to stop and thereby limit the first movement of the sleeve member along the recessed portion.

17. (canceled)

18. The surgical handle in accordance with claim 1 wherein the upper portion of the sleeve comprises an outwardly divergent section with a greater diameter relative to the diameter of the lower section of the sleeve that is received into the gripping member.

19. The surgical handle in accordance with claim 1 further comprising a head mounted onto the distal portion of the body.

20. The surgical handle in accordance with claim 19 wherein the head further comprises a cap that is arranged to be movable relative to the distal portion of the body and/or the head.

21. The surgical handle in accordance with claim 1 wherein the first movement comprises axial movement of the in-use upper portion of the sleeve away from the distal portion of the body and wherein the second movement comprises axial movement of the in-use upper portion of the sleeve away towards the distal portion of the body.

22. The surgical handle in accordance with claim 1 wherein the gripping member is elongate and comprises a middle portion extending between two end portions wherein cross-sectional diameter of the middle portion is greater than the cross sectional diameter of any one of the end portions.

23. The surgical handle in accordance with claim 1 wherein the proximal end portion comprises an inner cavity that is adapted to receive an end of shaft having a D-shaped profile such that a flat portion of the shaft engages a corresponding flat portion along the inner surface defining the cavity of the body.
